(19) United States
(12) Patent Application Publication
(10) Pub. No.: US 2025/0331778 A1
(43) Pub. Date: Oct. 30, 2025

(54) Systems and Methods for Hemodynamic Monitoring Using a Computational Surrogate for Heart Position

(71) Applicant: Becton, Dickinson and Company, Franklin Lakes, NJ (US)

(72) Inventors: Boris Reuderink, Amersfoort (NL); Jeroen Van Goudoever, Amsterdam (NL); Hans Jean Paul Kuijkens, Amsterdam (NL)

(21) Appl. No.: 19/189,938

(22) Filed: Apr. 25, 2025

Related U.S. Application Data
(60) Provisional application No. 63/639,342, filed on Apr. 26, 2024.

(51) Int. Cl. A61B 5/00; A61B 5/021; G16H 40/63

(52) U.S. Cl. CPC A61B 5/721; A61B 5/021; A61B 5/6824; A61B 5/6826; A61B 5/742; G16H 40/63

(57) ABSTRACT

Systems and methods for correcting sensed hemodynamic data are provided. Sensed hemodynamic data can be affected by hydrostatic forces and thus a correction is applied based on vertical position of where the site of sensing is being performed. The correction can be a computationally determined.

### TECHNOLOGICAL FIELD

The disclosure is generally directed to systems and methods for hemodynamic monitoring, and more specifically for systems and methods that adjust sensed hemodynamic parameters to heart level.

### BACKGROUND

Continuous noninvasive blood pressure monitors enable real-time measurement of blood pressure waves and derived hemodynamic parameters. Popular techniques utilized include volume clamping and tonometry. Other emerging techniques include pulse wave transit time, pulse arrival time, and capacitive-based pressure sensors. Each technique has their benefits and drawbacks but generally continuously monitors blood pressure by placing one or more sensors on an extremity of a patient to sense physiology related to blood pressure and heart beats. In each case, the system acquires a blood pressure at the site of sensor placement. The location of sensor relative to the heart can affect blood pressure readings due to changes in hydrostatic pressure, as gravity affects the local blood pressure.

### SUMMARY

Systems and methods for continuous hemodynamical measurement can comprise utilization of a sensor system on a patient. The hemodynamic data sensed by the sensor can be corrected based on vertical position relative to the heart. A computational method can compute a correction based on the hemodynamic data yielded by the sensor system. A computational method can further identify that the vertical position of the sensor has changed and then can compute an updated correction based on the hemodynamic data yielded by the sensor system. The correction can be continually updated as sensor position changes and can be utilized to continually correct sensed hemodynamic parameters.

In some implementations, a real-time method for correcting sensed hemodynamic data comprises receiving, using a hemodynamic monitoring system, sensor signals acquired from a sensor system upon a site of sensing.

In some implementations, a real-time method for correcting sensed hemodynamic data comprises determining, using the hemodynamic monitoring system, a set of sensed hemodynamic parameters derived from the sensor signals.

In some implementations, a real-time method for correcting sensed hemodynamic data comprises computing, using a hemodynamic monitoring system, a correction that accounts for vertical position of the sensor system.

In some implementations, a real-time method for correcting sensed hemodynamic data comprises continually correcting, using the hemodynamic monitoring system, a sensed hemodynamic parameter using the correction that accounts for vertical position of the sensor.

In some implementations, the step of computing, using the hemodynamic monitoring system, a correction that accounts for vertical position of the sensor system further comprises: entering, using the hemodynamic monitoring system, a set of sensed hemodynamic parameters into a trained computational model to yield a corrected hemodynamic parameter.

In some implementations, the step of computing, using the hemodynamic monitoring system, a correction that accounts for vertical position of the sensor system further comprises: utilizing, using the hemodynamic monitoring system, the corrected hemodynamic parameter within an equation of invariant inputs to determine the correction that accounts for vertical position of the sensor system. The inputs include the corrected hemodynamic parameter and a sensed hemodynamic parameter. The corrected hemodynamic parameter and the sensed hemodynamic parameter are the same hemodynamic parameter.

In some implementations, the equation of invariant inputs is:

$$vHP + bias = sHP + vCorr$$

wherein vHP is the corrected hemodynamic parameter, sHP is a sensed hemodynamic parameter, and vCorr is the correction; wherein vHP and psHP are the same hemodynamic parameter.

In some implementations, vHP is vMAP and sHP is sMAP.

In some implementations, the step of computing, using the hemodynamic monitoring system, a correction that accounts for vertical position of the sensor system further comprises: receiving, using the hemodynamic monitoring system, motion detector signals, wherein the motion detector signals indicate a vertical position of the sensor system relative to heart level.

In some implementations, the step of computing, using the hemodynamic monitoring system, a correction that accounts for vertical position of the sensor system further comprises: determining, using the hemodynamic monitoring system, the correction using the vertical position of the sensor system relative to heart level.

In some implementations, a real-time method for correcting sensed hemodynamic data comprises identifying, using the hemodynamic monitoring system, a vertical motion of the sensor system corresponding to a change in vertical position of the sensor system.

In some implementations, a real-time method for correcting sensed hemodynamic data comprises updating, using the hemodynamic monitoring system, the correction that accounts for vertical position of the sensor system.

In some implementations, the step of updating, using the hemodynamic monitoring system, the correction that accounts for vertical position of the sensor system comprises: reentering, using the hemodynamic monitoring system, an updated set of sensed hemodynamic parameters into the trained computational model to yield an updated corrected hemodynamic parameter.

In some implementations, the step of updating, using the hemodynamic monitoring system, the correction that accounts for vertical position of the sensor system comprises: utilizing, using the hemodynamic monitoring system, the updated corrected hemodynamic parameter within the equation of invariant inputs to determine the correction that accounts for vertical position of the sensor system. The inputs include the updated corrected hemodynamic parameter and an updated sensed hemodynamic parameter. The updated corrected hemodynamic parameter and the updated sensed hemodynamic parameter are the same hemodynamic parameter.

In some implementations, the step of identifying, using the hemodynamic monitoring system, a motion of the sensor system comprises: receiving, using the hemodynamic monitoring system, motion detector signals, wherein the motion detector signals indicate that an amount of vertical motion is greater than a threshold.

In some implementations, the motion detector signals indicate that a change of gravitational force is greater than a threshold.

In some implementations, the motion detector signals are derived from a motion detector that is associated with the sensor system.

In some implementations, the step of identifying, using the hemodynamic monitoring system, a motion of the sensor system comprises:

In some implementations, detecting, using the hemodynamic monitoring system, the vertical motion of the sensor system from a computational model trained to detect a change in sensed hemodynamic parameters in response to vertical repositioning of the sensor system.

In some implementations, the step of updating, using the hemodynamic monitoring system, the correction that accounts for vertical position of the sensor system comprises: receiving, using the hemodynamic monitoring system, motion detector signals, wherein the motion detector signals indicate a change in vertical position of the sensor system relative to heart level.

In some implementations, the step of updating, using the hemodynamic monitoring system, the correction that accounts for vertical position of the sensor system comprises: determining, using the hemodynamic monitoring system, the correction using the change of vertical position of the sensor system relative to heart level.

In some implementations, the sensor system comprises at least one of: an applanation tonometer, a pressure cuff, a photoplethysmograph, a peripheral arterial line, one or more electrode leads, a pulse oximeter, or a capacitance-based pressure sensor.

In some implementations, the step of determining, using the hemodynamic monitoring system, a set of sensed hemodynamic parameters comprises: performing arterial tonometry, performing volume clamping, performing catheter-based hemodynamic monitoring, performing pulse wave transit time or pulse arrival time, performing photoplethysmography-based heart rate monitoring, or performing arterial pressure sensing via capacitance.

In some implementations, a real-time method for correcting sensed hemodynamic data comprises computing, using the hemodynamic monitoring system, a downstream hemodynamic parameter from a corrected sensed hemodynamic parameter.

In some implementations, the downstream hemodynamic parameter is a centralized hemodynamic parameter.

In some implementations, the centralized hemodynamic parameter is blood pressure within the ascending aorta.

In some implementations, a real-time method for correcting sensed hemodynamic data comprises generating, using the hemodynamic monitoring system, a waveform utilizing corrected sensed hemodynamic parameters.

In some implementations, a real-time method for correcting sensed hemodynamic data comprises generating, using the hemodynamic monitoring system, a waveform utilizing downstream hemodynamic parameters. a real-time method for correcting sensed hemodynamic data comprises displaying the waveform on a display of the hemodynamic monitoring system.

In some implementations, the hemodynamic monitoring system is a clinical use system, a portable system, or a wearable device.

In some implementations, the hemodynamic monitoring system excludes a paired sensor system for establishing vertical position of the sensing system relative to a heart based on a difference of vertical position between each sensor of the paired sensor system to compute the correction.

In some implementations, the hemodynamic monitoring system excludes one or more sensors positioned at heart level for establishing the vertical position of a heart to compute the correction.

In some implementations, the hemodynamic monitoring system excludes the use of a central blood pressure sensor positioned at a non-thoracic location to compute the correction.

In some implementations, a hemodynamic monitoring system for real-time correction of sensed hemodynamic data comprises a sensor system.

In some implementations, a hemodynamic monitoring system for real-time correction of sensed hemodynamic data comprises a computational processing system in digital connection with the sensor system

In some implementations, the computational processing system comprises a processor system and memory system comprising one or more applications that can direct the processor system to perform the various computational methods described herein.

### BRIEF DESCRIPTION OF THE DRAWINGS

The description and claims will be more fully understood with reference to the following figures and data, which are presented as examples of the disclosure and should not be construed as a complete recitation of the scope of the disclosure.

FIG. 1 provides an example of a sensor system with a heart reference sensor for continuous blood pressure monitoring.

FIG. 2 provides an example of a sensor system for hemodynamic monitoring.

FIG. 3 provides an example of a method for adjusting continuous hemodynamic measurement.

FIG. 4 provides an example of a computational method for correcting hemodynamic monitoring parameters based on a computational surrogate for heart position.

FIG. 5 provides an example of how to correct a sensed hemodynamic data via a computational surrogate.

FIG. 6A provides an example of a computational processing system for continuous hemodynamic monitoring.

FIG. 6B shows an example hemodynamic monitoring system that integrates both pressure sensing and motion detection, according to some embodiments.

FIG. 6C illustrates an example method for correcting blood pressure measurements based on beat-level processing and motion detection, in accordance with some embodiments.

FIGS. 7A to 7C provide examples of continuous hemodynamic data comparing a classical heart reference system with a computational surrogate for heart position.

### DETAILED DESCRIPTION

The current disclosure details systems and methods to perform real-time continuous hemodynamic monitoring using a sensor system. The systems and methods improve hemodynamic monitoring by computing a “heart-level correction” of hemodynamic parameters sensed by the sensor system, and adjusting the hemodynamic data to compensate for hydrostatic pressure changes that arise due to the vertical difference between the local blood pressure at the site of the sensor and at the pressure at the heart. These hydrostatic differences can cause significant distortion in blood pressure readings if uncorrected. The hydrostatic differences can correspond to a pressure change of about 0.77 mmHg per centimeter of vertical displacement. Traditionally, various methodologies directly detected position of the sensor system in order to compensate for vertical differences. As described herein, a computational surrogate is utilized to correct sensed hemodynamic parameters to parameters at heart level, reducing the need for extra sensors and cables while still achieving robust hemodynamic monitoring with hydrostatic pressure compensation. The systems and methods described can be applied to any modality that utilizes a sensor system for hemodynamic monitoring, as the compensation performed is related the vertical differences in arterial pressures between the heart level and the actual site where sensing is performed.

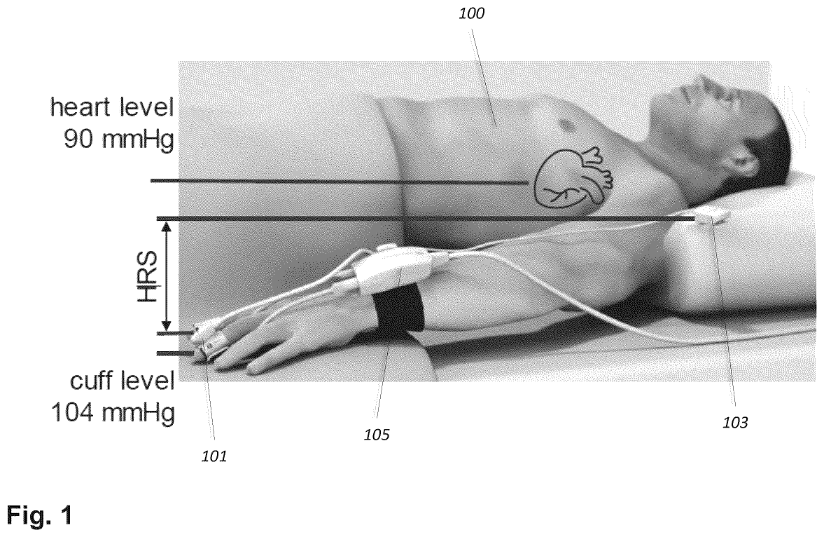

Provided in FIG. 1 is an example of a classical heart reference sensor utilized with a hemodynamic monitoring system comprising a sensing system. Patient 100 is having their blood pressure monitored via a sensor system 101 attached to the digits and wrist of the patient. As shown, the vertical level of the hand of patient 100 with attached sensor system 101 is below the vertical level of the heart, and thus a higher pressure would be sensed relative to the heart level. Conversely, if the vertical level hand of patient 100 with attached sensor system 101 was above the vertical level of the heart, a lower pressure would be sensed relative to the heart level. In this classical heart reference sensor (HRS) system, a first sensor 103 is strategically placed at or near the level of the patient's heart and a second sensor 105 is provided at or near sensor system 101. The vertical distance between first sensor 103 and second sensor 105 can continually or continuously be assessed by and utilized to correct the continuous hemodynamic monitoring being performed by sensor system 101. This ensures less variation in hemodynamic monitoring and better downstream applications. For instance, continuous hemodynamic monitoring can be displayed on a display screen, utilized for further calculation of hemodynamic parameters (e.g., central blood pressure within the ascending aorta), and providing alerts based on parameter thresholds and trending. Such alerts may include values and/or deviations in mean arterial pressure (MAP), virtual MAP (vMAP), diastolic/systolic values, and/or other arterial pressure values. Accordingly, reducing variation due to vertical position of sensing yields more consistency in hemodynamic monitoring and better patient outcomes.

Here, various implementations of a hemodynamic monitoring system can rely a computational surrogate for correcting real-time hemodynamic data as related to relative vertical position of the sensing system. Accordingly, in some implementations, a hemodynamic monitoring system excludes the use of a classical heart reference sensor system. In some implementations, a hemodynamic monitoring system excludes the use of a paired sensor system for establishing vertical position of the hemodynamic sensing system relative to a patient's heart based on a difference of vertical position between each sensor of the paired sensor system. And in some implementations, a hemodynamic monitoring system excludes the use of one or more sensors positioned at the heart level for establishing the vertical position of the patient's heart. In some implementations, a sensing system excludes the use of a hemodynamic sensor for tracking central hemodynamic parameters (e.g., a central blood pressure sensor), which may be positioned at central location or a peripheral location of the patient. For example, a sensing system may exclude a central blood pressure sensor positioned at a non-thoracic location. In some implementations, a sensing system excludes the combined use of a hemodynamic sensor for tracking central hemodynamic data with a hemodynamic sensor for tracking peripheral hemodynamic data.

In some implementations, a hemodynamic monitoring system comprises a motion detection system for capturing motion of a site where sensing is occurring. In some implementations, motions can be detected from the captured data itself. For example, a predictive computational model can be trained to utilize features from captured data to predict when vertical motion of a sensor system has occurred. In some implementations, the captured data is utilized as data input within a computational model as a computational surrogate to correct for vertical position. And in some implementations, detection of vertical motion of a sensing system is utilized to trigger computational steps to adjust a vertical-position correction. For example, when a patient's arm is raised or lowered, detected acceleration and inferred position changes can serve as a real-time signal to update the height offset correction term, even in the absence of absolute position measurements.

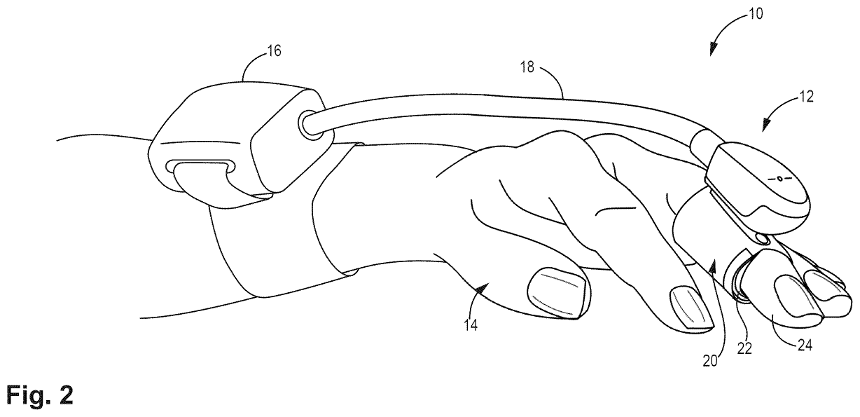

An example of a sensing system 12 attached to a hand 14 is provided in FIG. 2. Although the sensing is depicted as fitted onto fingers and wrist, it should be understood that hemodynamic sensing can be performed at any location that can have hemodynamic parameters sensed by a sensor system. In some implementations, the sensor system is located a non-heart level vertical position. In some implementations, the vertical position of a sensor system is adjustable relative to a heart level vertical position. A sensor system can be centrally placed (e.g., at a thoracic location) or peripherally placed (e.g., at a non-thoracic location). Examples of peripheral locations that can be utilized include (but are not limited to) an arm, a finger, a thumb, a wrist, an ankle, a leg, a toe, an ear, a temple, etc. A sensor system can be invasive (including partially invasive and minimally invasive) or noninvasive.

As shown in the example of FIG. 2, sensor system 12 is a non-invasive hemodynamic sensor configured to acquire arterial blood pressure measurements through volume clamping. Sensor system 12 can include housing 16, connector 18, cuff 20, and pressurizable bladder 22. In the illustrated example, cuff 20 is a ring or similar structure surrounding or bracketing finger 24 of hand 14, while housing 16 is a wrist-mounted device coupled to cuff 20 via connector 18. In the most general case, however, sensor system 12 can differ substantially from the layout illustrated in FIG. 2. Sensor system 12 can, for example, include multiple separate connectors 18 between elements attached to finger 24 (e.g. cuff 20), and/or can relocate housing 16 to other locations (e.g. integrated with cuff 20, or separately disposed at another location), including various configurations described herein.

Cuff 20 can be configured to fit on a variety of body appendages, including an arm, a finger, a thumb, a wrist, an ankle, a leg, a toe, an ear, a temple, etc. In the illustrated example, cuff 20 surrounds a sensing region of a finger 24 of hand 14. At least one artery 26 passes through the sensing region. Cuff 20 also anchors pressurizable bladder 22, which can for example be an expandable annular fluid bladder fed by a fluid line included within connector 18, or from another source. Generally, fluid utilized to inflate a bladder can be air. In the most general case, however, pressurizable bladder 22 can be any sort of mechanism suited to apply pressure to finger 24 based on control as described below. Sensor system 12 and hand 14 together make up combined physical system 10 (sometimes referred to as a plant or plant system) responsive both to changes in the patient and change in control of sensor system 12.

Sensor system 12 also comprise a light emitter and detector which can be disposed upon cuff 20. The light emitter can be configured to emit light of one or more discrete wavelength bands onto an artery within finger 24 and the light detector can be configured to detect reflection and refraction of said emitted light. The light emitter and detector system can be utilized to perform photoplethysmography, pulse oximetry, or other sensing activities for acquiring or deriving hemodynamic parameters, especially blood pressure parameters.

A motion detection system can be utilized with sensor system 12. For example, a camera system (e.g., IR camera) can be positioned near and pointed towards sensor system 12, recording its movements. A marker for improving movement detection by a camera system may be incorporated into sensor system 12 such that the camera can easily track movement of the marker. In another nonlimiting example, a sensor system includes a motion detection sensor, such an accelerometer, a velocity sensor, a gyro sensor, or any other sensor configured to detect self-movement and is attachable to the appendage. The motion detector can be associated with or incorporated into the sensing system at or near the site of sensing such that it can detect the movement of that site. Referring back to FIG. 2, a motion detector can be incorporated into sensor system 12, cuff 20, connector 18, housing 16, or independently attached to the appendage. Alternatively, a motion detection system can lack a camera system and/or motion detection sensor and instead predict motion from the captured hemodynamic data.

For example, an accelerometer can be incorporated into and/or otherwise associated with the sensor system 12 to detect changes in orientation or position of the patient's appendage (e.g., hand, finger) relative to the heart level. Such changes may result from patient movement or positional shifts in the hospital bed (e.g., tilting and/or rotating the bed during surgery or recovery), which may alter the hydrostatic pressure gradient between the heart and the measurement site. Upon detecting a change in the appendage's orientation and/or elevation, the system may initiate a correction to adjust the measured blood pressure to reflect the pressure at heart level.

In some embodiments, the system continuously acquires signals from the motion detector (e.g., accelerometer, gyroscope). The system can also obtain hemodynamic data such as systolic pressure, diastolic pressure, mean arterial pressure (MAP), and/or derived features, such as maximum dP/dt. These data can be used by the system to estimate the likely height difference between the pressure sensing site and the anatomical level of the heart. When motion is detected by the motion detector, the system may infer that a positional change has occurred and use the motion data as a gating signal to trigger recalibration and/or correction of the MAP values.

The system may calculate a virtual mean arterial pressure (vMAP). The vMAP can be generated from a model, such as a trained model. The model can use heart rate, one or more cuff pressure values (e.g., systolic, diastolic, MAP, etc.), differences of said values (e.g., systolic minus diastolic), slopes of values that may be derived such as slope of pressure over time (e.g., max dP/dt), and/or other values. The vMAP can be generated for each beat. In some embodiments, the vMAP may represent a cleaned, smoothed MAP estimate across multiple beats. Additionally or alternatively, the system can determine the standard deviation of vMAP for each beat. The system may reject noisy and/or unreliable beats. In response to detected motion events, the system can update a virtual heart reference sensor (vHRS) based on the vMAP value. Between motion events, the vHRS remains static and is used to maintain an invariant relationship such as vMAP+bias=cMAP+vHRS, where cMAP is the measured cuff MAP and the bias term accounts for prior known offsets. Reference to sMAP below may correspond to cMAP.

In some embodiments, the system may incorporate a machine learning model trained to detect persistent changes in MAP that are not attributable to physiological variability alone. For example, if the system observes a consistent increase or decrease in blood pressure following a detected positional change, it may learn (e.g., update the model) to associate that pattern with an upward or downward shift in the measurement site relative to heart level. In some cases, without motion detection data, such changes might be mistaken for true changes in patient status. Thus, the motion detector can improve a trained model to attribute such variation to a hydrostatic pressure shift and update the model accordingly.

In some embodiments, the system uses acceleration and/or rotation data to detect changes in the orientation of the sensor relative to gravity. As an example, the system may identify a change in x- and/or y-axis accelerometer signals to determine that the sensor (and, thus, the clamp) has rotated. For instance, when the surgical bed is rotated or tilted, the accelerometer may detect a corresponding change in the signal relationship between axes, indicating that the height of the pressure measurement site (e.g., the patient's hand) has changed relative to heart level. This rotational cue can act as a trigger to initiate the algorithmic correction. In some embodiments, without a detectable orientation shift from the accelerometer, the correction algorithm can remain gated off, thus reducing a risk of introducing noise and/or overcorrection due to physiological variation, patient-specific blood pressure drift, and/or sensor artifacts.

The system may incorporate other safeguards, such as user-initiated overrides and/or contextual filters. For example, when a known perturbation occurs, such as administration of a fluid bolus (which may cause a legitimate and sudden change in blood pressure and/or motion), a user may press a button to signal the system to temporarily disregard algorithmic corrections. This temporary override may help avoid misinterpretation of the event as a height-induced pressure shift. Additionally or alternatively, the system may include an interface button that can allow recalibration of the virtual MAP to heart level to be in line with clinician-confirmed values. In some embodiments, the system may compute and/or display a quality metric (e.g., vHRS confidence score) derived from the variability and/or agreement between the measured vMAP and a plurality of trained model predictions. For example, if the vMAP for a given beat falls outside a defined standard deviation range relative to the trained ensemble, the system may reject the data and pause any updates to the correction factor.

Sensor system 12 is intended to be a nonlimiting example and thus other configurations can be utilized. A sensor can be any sensor capable of acquiring hemodynamic data, especially blood pressure parameters, such that it can acquire or derive a continuous hemodynamic pressure waveform. The sensor system can be non-invasive, minimally invasive, partially invasive, or fully invasive. Examples of sensors and their application include (but are not limited to): an applanation tonometer for performing arterial tonometry, a pressure cuff and photoplethysmograph for performing volume clamping, a peripheral arterial line for performing catheter-based hemodynamic monitoring, an internal pressure transducer for performing internal hemodynamic monitoring, one or more electrode leads and pulse oximeter for performing pulse wave transit time or pulse arrival time, a photoplethysmograph for performing photoplethysmography-based heart rate monitoring, and a capacitance-based pressure sensor for performing arterial pressure sensing via capacitance. In some implementations, the sensor system is a clinical use device intended for use within a professional healthcare facility, such as a catheter-based system, internal system, or the example portrayed in FIG. 2. In some implementations, the sensor system is a portable device intended for use in any environment, especially outside a professional healthcare facility (e.g., at home). In some implementations, the sensor system is a wearable device intended for everyday use, for ambulatory use, or for use during exercise. The sensor system may incorporate a motion detector, such as an accelerometer or gyroscope, to detect orientation changes relative to gravity and so determine a change in hydrostatic pressure due to vertical displacement of the sensing site. Furthermore, a motion detection system can be utilized with any sensor system, but especially a sensor system that is capable of adjusting vertical height relative to heart level.

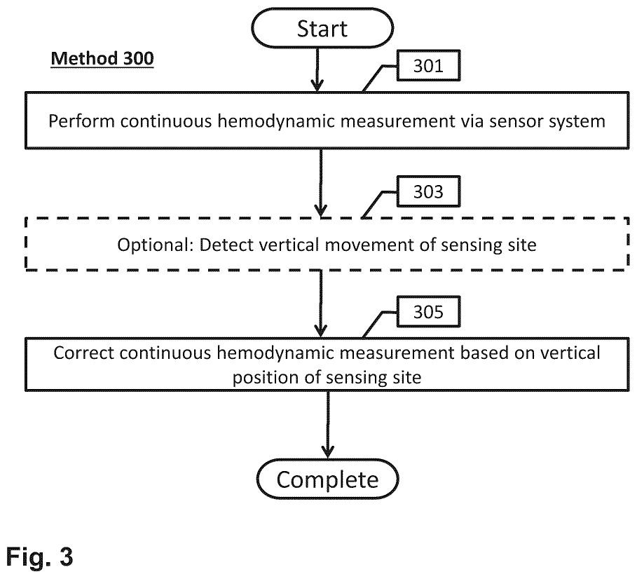

Provided in FIG. 3 is an example of a method for performing continuous hemodynamic measurements correcting for vertical position relative to heart level. Method 300 can begin by performing (301) continuous hemodynamic measurement on a recipient via a sensor system. Any sensor system for capturing or deriving hemodynamic parameters can be utilized. The sensor can be noninvasive (e.g., blood pressure cuff) or can be invasive (e.g., internal pressure transducer). The continuous hemodynamic measurement can be any hemodynamic measurement that can be affected by gravitational forces, such as (for example) blood pressure, systolic arterial pressure, diastolic arterial pressure, and mean arterial pressure. Virtually all hemodynamic parameters related to pressure are affected by gravitational forces, which can result in a need to correct measurements acquired at a non-heart level vertical position to achieve greater consistency. Such corrections may be based on a determination of the vertical offset between the heart and the measurement site. The vertical offset may be calculated in response to orientation and/or motion data collected by the sensor system.

Any site that can be used for sensing and can be at a different vertical position than the heart benefits from correction, especially at sites where vertical movement can occur. Even subtle movements, such as (for example) lifting of an appendage or adjustment of a hospital bed can affect hemodynamic sensing. For example, a change in x- and/or y-axis accelerometer data can indicate that the measurement site has moved relative to gravity. This determination may trigger an adjustment to the measured blood pressure. Accordingly, the method can be performed on any central or peripheral site, including (but not limited to) within an artery, upon an arm, upon a finger, a thumb, upon a wrist, upon an ankle, upon a leg, upon a toe, upon an ear, upon a temple, etc.

In many implementations, performing continuous hemodynamic measurement results in capturing a waveform (e.g., arterial pressure waveform), which can be displayed on a monitor. In many implementations, continuous hemodynamic measurements are utilized to derive a variety of other hemodynamic parameters. Accordingly, vertical level of the sensor system or vertical movement thereof can affect various downstream computations, visualizations, and assessments. The system may compute a virtual mean arterial pressure (vMAP), a smoothed MAP estimate, and/or deviations associated with measurements during movement.

Method 300 can also optionally detect or predict (303) vertical movement of a site where sensing is occurring. Vertical movement can be detected by monitoring the movement of the sensor systems using a variety of mechanisms, such as a camera system or an attached motion detector. In some embodiments, motion detector signals are continuously acquired. Changes in acceleration and/or rotation may be used as gating events to initiate recalibration of measured pressure values. Alternatively, because a change in vertical position can result in a change of hemodynamic measurement capture, captured hemodynamic data can be utilized as input within a predictive computational model to detect movement

Method 300 can also correct (305) the continuous hemodynamic measurement based on vertical position of the sensing site. In some implementations, a correction is determined in real-time based on the sensed hemodynamic measurements, which can then be used to correct the hemodynamic measurements for vertical position. Furthermore, when vertical movement of the sensing site is detected, the correction can be adjusted to ensure appropriate correction of the hemodynamic measurements for vertical position is maintained. In some instances, a computational correction of the continuous hemodynamic measurement acts as a surrogate of a heart reference sensor system. In some instances, computational correction of the continuous hemodynamic measurement is performed in lieu of utilizing a heart reference sensor system.

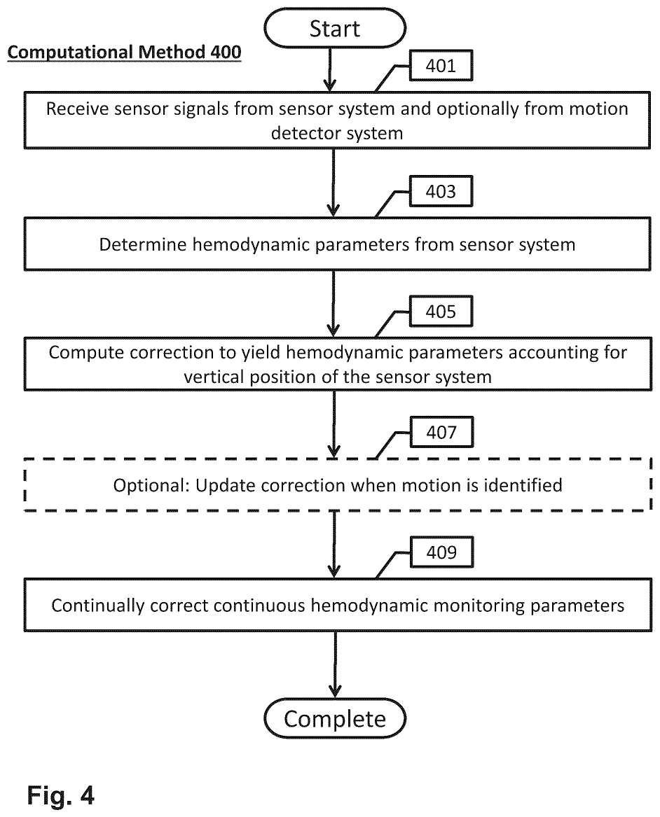

Provided in FIG. 4 is a computational method for correcting continuous hemodynamic monitoring parameters. Computational Method 400 can be performed utilizing a computational device equipped with instructions for performing the method. The computational device can be part of a hemodynamic monitoring system. The computational device can be in connection with a hemodynamic measurement sensing system and optionally a motion detection system such that it can receive data from said systems in real time. The computational device can perform the steps of Computational Method 400 in real time such that it can continually correct hemodynamic parameters acquired by the sensing system. Corrected hemodynamic parameters can be displayed on a display screen, utilized in further downstream computations, and/or utilized to provide medical alerts in real time, enhancing the utility of hemodynamic monitoring using hemodynamic measurement sensing systems. In some embodiments, the system can additionally or alternatively compute and/or display a confidence score for corrected values (e.g., vMAP, vHRS). The confidence score may be derived from variability in the sensed signals and/or agreement with trained model outputs.

Computational Method 400 can receive (401) sensor signals from a sensor system and optionally from a motion detector system. As described herein, virtually any of a number of sensor systems for acquiring hemodynamic parameters can be utilized. The sensor can be non-invasive or invasive; central (e.g., thoracic) or peripheral (e.g., non-thoracic). Generally, the sensor system can acquire sensor data such as capacitance-based pressure measurements, photoplethysmography, tonometry, light signals, electronic signals, each of which can be utilized by various methodologies to yield hemodynamic data. Further, a motion detector system can optionally be utilized to detect occurrences of vertical motion. Signals of from motion detector system can be acquired via a camera system or a motion detector. For example, an accelerometer may be used to detect orientation and/or elevation shifts of the patient's appendage relative to heart level, including changes induced by movement or surgical bed tilting. Generally, the motion detector system can be used to identify when the site of sensing changes vertical position. Or, alternatively, a motion detector system can be a computational-based system that detects motion from acquired or derived hemodynamic measurements.

Computational Method 400 can also determine (403) hemodynamic parameters from the sensor system. The various methodologies of hemodynamic monitoring can utilize the acquired signals to continuously derive hemodynamic parameters. For example, the volume clamp method acquires photoplethysmography via light signal data to determine arterial volume within the appendage at the site of sensing. Because each heartbeat results in expansion and contraction of the arterial volume in accordance with systolic pressures and diastolic pressures, a blood pressure cuff can be utilized in conjunction with the photoplethysmograph to continuously determine arterial pressure. To do so, the blood pressure cuff is continuously inflated and deflated with a fluid such that the arterial volume within the appendage stays constant. When the arterial volume stays constant, the pressure within the cuff is matching the arterial systolic pressures and diastolic pressures, thus providing arterial pressure readings from the sensor system. In some implementations, derived values such as maximum dP/dt and/or other values disclosed herein may be calculated/generated from beat-to-beat signal analysis. Further description of examples of various systems and methodologies to yield hemodynamic parameters from various sensor systems are provided in X. Quan et al., Sensors (Basel). 2021 Jun. 22; 21 (13): 4273, the disclosure of which is hereby incorporated by reference.

Computational Method 400 can also compute (405) a correction to yield hemodynamic parameters accounting for vertical position of the sensor system. The hemodynamic parameters acquired by the sensor system are affected by relative vertical position. More specifically, blood pressure at a sensing site is determined in part by hydrostatic pressure. Effects of hydrostatic pressure is lesser when a sensor system is at a higher vertical position, and greater when a sensor system is at a lower vertical position. To account for these differences, it is ideal to standardized pressure readings to a single vertical position, reducing variation and improving downstream assessments and computations. Any vertical position can be selected for standardization. In many instances, it is a goal to standardize hemodynamic parameters to heart level.

In several implementations, one or more computational models are trained to predict a corrected hemodynamic parameter (e.g., predicting a corrected pressure parameter from sensed hemodynamic parameters). To perform training, a classical heart reference sensor can be utilized to obtain corrected hemodynamic parameters. The data prior to correction (i.e., sensed hemodynamic data) is utilized as input within the computational and is associated with corrected hemodynamic parameters obtained using a classical heart reference sensor. The computational model can be trained to predict the corrected hemodynamic parameter from the input data. The model may be updated in response to determinations that a motion has occurred. For example, the model may learn to associate an upward and/or downward shift with associated motion patterns. Otherwise, the system may mistakenly associate such shifts with true changes in patient status. Additionally or alternatively, motion signals from a motion detector may be used as gating triggers for recalibration of the data and/or of updating of the model. Any predictive computational model for predicting corrected hemodynamic parameters from sensed sensor hemodynamic data can be utilized, such as neural networks and linear regression.

A computational model can be utilized to predict any of a number of various outputs, such as (for example) a corrected blood pressure, corrected systolic arterial pressure, a corrected diastolic arterial pressure, or a corrected mean arterial pressure. Further, several sensed hemodynamic data features can be utilized as input, such as (for example) a sensed blood pressure, features derived from a sensed hemodynamic waveform, a sensed stroke volume, a sensed pulse wave transit time, a sensed pulse arrival time, a sensed systolic arterial pressure, a sensed diastolic arterial pressure, a sensed mean arterial pressure, a sensed dP/dt, a sensed sys/dia pressure ratio, etc. In some implementations, the input features are polynomial features, especially when a regression model is utilized. To improve predictive function, input features should be fairly robust and insensitive to perturbations that may arise during continuous hemodynamic monitoring. For example, noisy and/or anomalous beats may be automatically rejected if they exceed model-based thresholds on beat-to-beat variability and/or if they are outside a threshold alignment with ensemble predictions (e.g., beyond one or two standard deviations). In accordance with several implementations, a computational model is designed to utilize several feature inputs to predict a singular output, or a defined set of outputs. In some implementations, several distinct computational models are individually trained and then combined to yield an ensemble model that yields a combined output (which can further yield a standard deviation and other statistical indicators).

In one illustrative example, a computational model utilizes polynomial input features to predict a corrected mean arterial pressure (i.e., vMAP where v stands for virtually predicted). Polynomial input features can comprise one or more of: heart rate, sensed systolic pressure, sensed diastolic pressure, sensed mean arterial pressure, sensed dP/dt, and sensed sys/dia pressure ratio. Additionally or alternatively, the system can determine a standard deviation of vMAP for each beat. The standard deviation may be based on values measured for a set number of beats. The system may reject noisy and/or unreliable beats that fall outside the standard deviation (or some other threshold).

To compute a correction, an equation with invariant inputs can be utilized. In some implementations, the following equation is utilized:

$$vHP + bias = sHP + vCorr \qquad EQ . 1$$

where vHP is a virtually predicted hemodynamic parameter predicted from a computational model, sHP is a sensed hemodynamic parameter, and vCorr is the correction (i.e., the virtually computed correction). Notably, the predicted hemodynamic parameter and the sensed hemodynamic parameter are the same hemodynamic parameter (e.g., both mean arterial pressure) Accordingly, vCorr can be computed by using vHP, bias, and sHP. Bias can initially be set to any number (e.g., zero). Once vCorr is computed, vHP, sHP, and vCorr can be kept invariant, allowing for bias corrections. In some implementations, bias is corrected using a low pass filter, such as an exponential filter, to remove noise and perturbations that may arise during continuous hemodynamic monitoring. Computed vCorr can be used to correct sensed hemodynamic data. For example, an arterial pressure waveform as sensed by the sensor system can be corrected using vCorr.

Referring back the illustrative example in a computational model is utilized to compute vMAP, the following equation can be utilized:

$$vMAP + bias = sMAP + vCorr \qquad EQ . 2$$

where vMAP is a virtually predicted mean arterial pressure predicted from a computational model, sMAP is a sensed mean arterial pressure, and vCorr is the correction (i.e., the virtually computed correction).

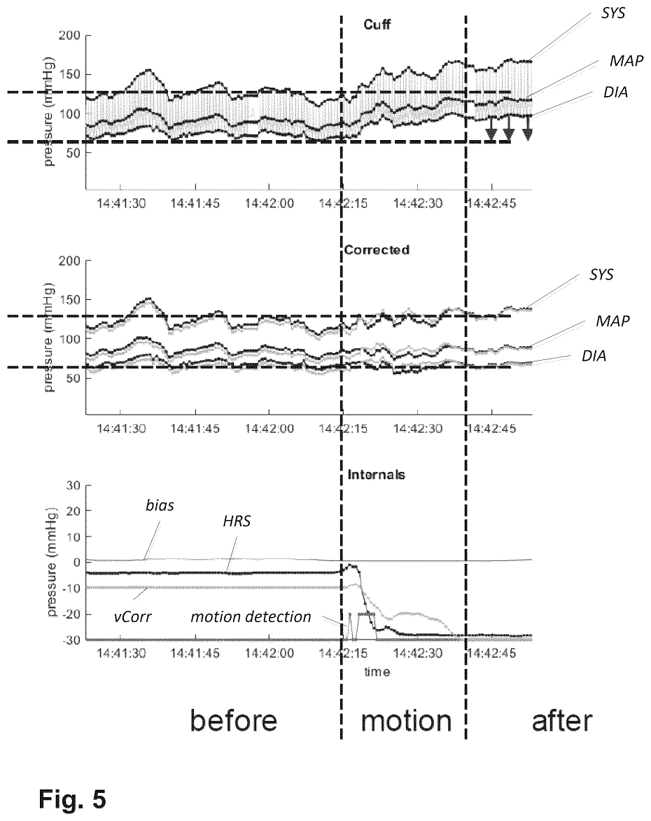

Computational Method 400 can also optionally update (407) a correction when motion is identified. As noted previously, a correction can remain invariant when the sensing site has remained unmoved. However, when motion is identified, the correction can be adjusted to account for the new vertical position of the sensing site. To update the correction, the correction is recomputed, which can be computed using EQ. 1 or EQ. 2. In some implementations, the computation of the correction is gated after motion is detected. The gate can be based on a period of time (e.g., 10 seconds) or a number of heartbeats (e.g., 10 heart beats). Once the gated period has ended, the computed correction can remain invariant until motion is detected again. For an example of a gated period, see FIG. 5 and accompanying description. In some embodiments, the motion event may trigger an update of vHRS based on the newly calculated vMAP to improve stability of the correction after repositioning.

In some implementations, motion is detected via a motion detection system, which can be a camera system, an attached motion detector, or a computational surrogate. In some implementations using a motion detection system, thresholds can be utilized to determine whether the motion requires the system to update the correction. In one illustrative example, the changes in gravitational force effected by the motion can be sensed (e.g., via an motion detector associated with the sensor system) or otherwise derived, and when the change of gravitational force is greater than a threshold, the process is triggered to update the correction. In another illustrative example, the amount of vertical motion can be monitored (e.g., via a camera system) or otherwise derived, and when the amount of vertical motion is greater than a threshold, the process is triggered to update the correct.

And in some implementations, a motion detection system detects motion via a computational surrogate using the sensed hemodynamic data as input. When a change in sensed hemodynamic parameters is identified (e.g., a global increase or decrease of pressure waveform data), a computational model can be used to determine whether this change in sensed hemodynamic parameters is due to repositioning of the sensing site or due to other factors (e.g., a cardiac event). For instance, a neural network can be trained to detect vertical motion from features derived from the sensed hemodynamic data. In some implementations, the sensed hemodynamic data features can be based on a data trend within an immediately prior period, such as (for example) the immediately prior 10 seconds or the immediately prior 10 heartbeats.

In some implementations, a motion detection system directly determines vertical position or changes in vertical position, which is then used to compute a correction. In some of such implementations, a motion detection system determines vertical position, which can be determined relative to heart level. Based on relative vertical position, a correction can be determined. In some of such implementations, a motion detection system determines changes in vertical position, which can be used to determine an adjustment to a correction. The system may initiate correction only in response to motion that indicates a change in hydrostatic gradient beyond a threshold amount. When adjusting the correction, the amount the correction is adjusted can be determined using the motion detection system or as described herein using EQ. 1 or EQ. 2.

Computational Method 400 can also continually correct (409) continuously sensed hemodynamic monitoring parameters. In some implementations, the sensed hemodynamic monitoring parameters are corrected to the heart level. Any periodicity can be utilized for continual correction. In some implementations, the sensed hemodynamic monitoring parameters are corrected each heartbeat. In some implementations, the amount of correction is gated (i.e., vCorr cannot be greater than an upper threshold or lesser than a lower threshold). For instance, if a sensor system is fitted onto a finger, the greatest possible distance away from the heart is typically less than 40 cm, and thus thresholds can be set accordingly. In some instances, when multiple computational models are utilized to determine vHP, and when the standard deviation is greater than threshold, an alternative vCorr is utilized, such as (for example) a prior computed vCorr. Various other safeguards can be utilized as well.

Provided in FIG. 5 is an example of correcting sensed hemodynamic data. In this example, a blood pressure cuff comprising a pressurizable bladder and a photoplethysmograph was utilized to continuously capture arterial blood pressure. The top panel of FIG. 5 shows the raw blood pressure data captured by the blood pressure cuff, including the systolic arterial pressure (SYS), the mean arterial pressure (MAP), and diastolic arterial pressure (DIA). The two dashed horizontal lines represent the average systolic and diastolic pressures, respectively. As can be seen at timepoint 14:42:15, the SYS, MAP, and DIA rise, indicating that the blood pressure cuff is sensing greater blood pressure. As shown in the bottom panel of FIG. 5, motion is detected at timepoint 14:42:15 and thus the increase in blood pressure is due to vertical repositioning of the site of sensing. The new vertical position results in the sensed pressures to remain elevated, and thus there is a need to adjust the correction of the sensed blood pressure. This observed increase may be correlated with motion data detected by a motion detection system (e.g., an accelerometer or gyroscope) that senses a change in orientation or position of the sensor relative to heart level.

The dashed vertical lines within FIG. 5 represent the gated period that the correction was recomputed. Accordingly, after the gated portion, the correction remains invariant. Shown in the bottom panel of FIG. 5 are two separately determined corrections. The darker shaded line represents a correction determined by the classical heart reference sensor (HRS) methodology (see FIG. 1). The lighter shaded line a correction determined by a computation surrogate (vCorr). As can be seen, both the classical HRS and surrogate vCorr adjust downwardly, resulting in a correction that lowers the sensed pressures. In some implementations, this gating is initiated based on a detected change in position (which may correspond to a change in vertical position) from the motion detection system.

The middle plane of FIG. 5 provides the corrected blood pressure, which has been corrected to the heart level. The darker shaded lines represent sensed blood pressure corrected via the classical HRS system. The lighter shaded lines sensed blood pressure corrected via a computational surrogate. As can be seen, the computational surrogate was able to detect motion and adjust a correction during a gated period accordingly, resulting in corrected blood pressure that is very representative of the HRS corrected blood pressure.

While specific examples of processes to correct sensed hemodynamic parameters are described above, one of ordinary skill in the art can appreciate that various steps of the process can be performed in different orders and that certain steps may be optional according to various implementations. As such, it should be clear that the various steps of the process could be used as appropriate to the requirements of specific applications. Furthermore, any of a variety of processes to correct sensed hemodynamic parameters appropriate to the requirements of a given application can be utilized in various implementations.

### Hemodynamic Monitoring System

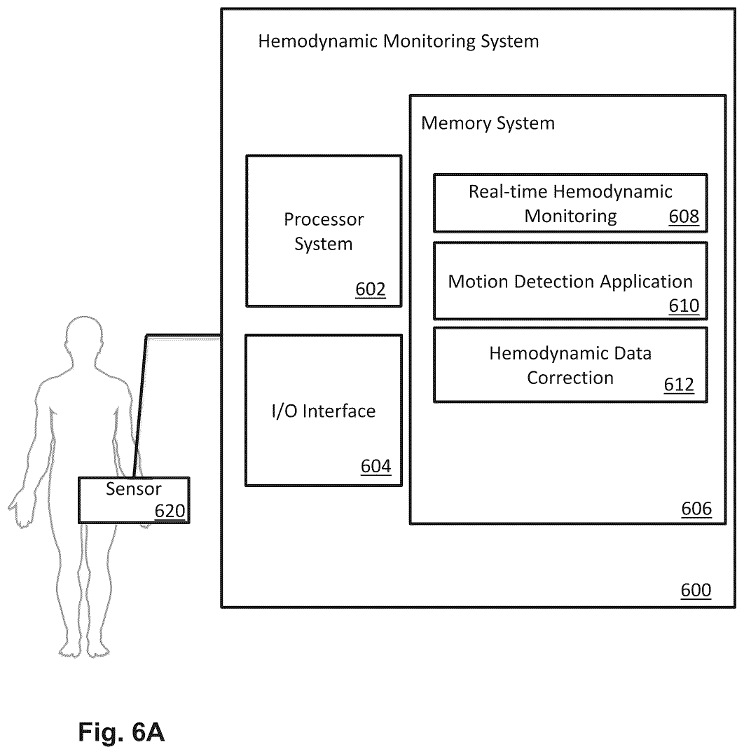

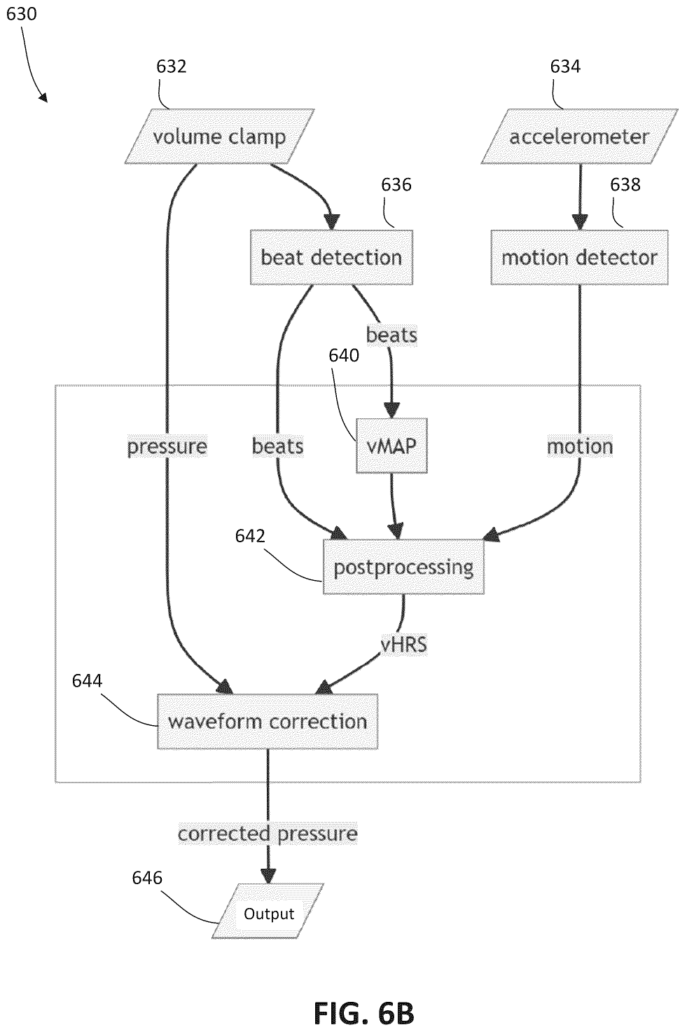

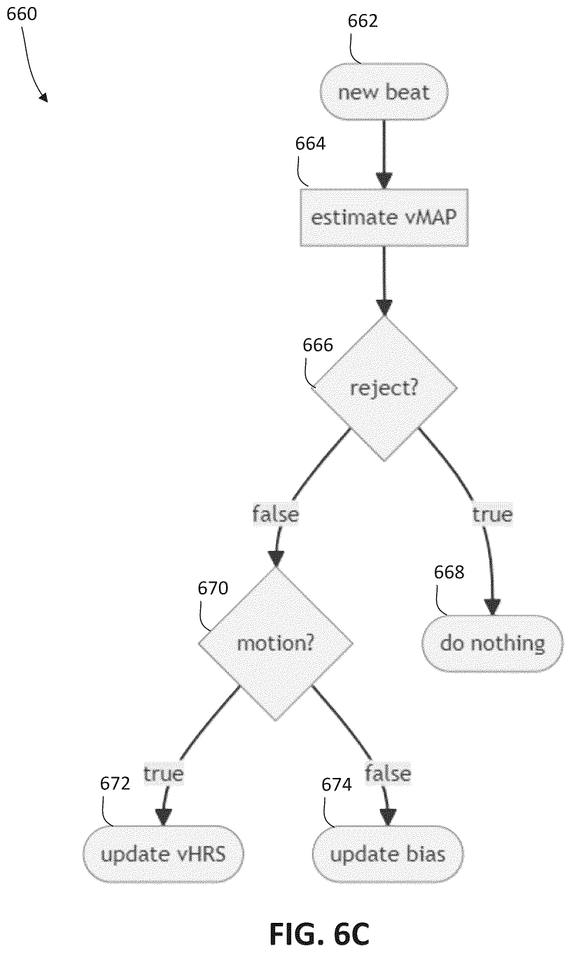

The various systems and methods of the current disclosure can be implemented or performed utilizing a hemodynamic monitoring system. Generally, the hemodynamic monitoring system includes a sensing system and a computational system. Provided in FIG. 6A is an example of a hemodynamic monitoring system 600 as would be utilized on an appendage of an individual. On the appendage is a sensor 620 that is capable of capturing sensor signals for computing hemodynamic parameters. Notably, sensor 620 can be any sensor for capturing hemodynamic measurements can be positioned at any location appropriate for that sensor. For example, the sensor 620 may include a photoplethysmography (PPG) sensor, a pressure transducer, and/or an accelerometer. Hemodynamic monitoring system 600 can be a clinical use system intended for use within a professional healthcare facility, a portable system intended for use for use in any environment, including environments outside a professional healthcare facility (e.g., at home), or a wearable device intended for everyday use, for ambulatory use, or for use during exercise. As can be readily appreciated, various configurations of a hemodynamic monitoring system are contemplated as appropriate for its intended use.

Sensor 620 can be in connection with hemodynamic monitoring system 600 such that data can be transmitted therebetween. Other systems as appropriate for sensing via sensor 620 can also be utilized, such as (for example) a fluid pump system, an electrocardiogram, and/or power supply. Sensor 620 can be connected via any means for transferring data, such as wired connection via cables or wires or wireless connection via Bluetooth, WiFi, etc.

Hemodynamic monitoring system 600 can comprise a computational system, comprising a processor system 602 for and I/O interface 604 for input and output of data, such as data communicated between hemodynamic monitoring system 600 and sensor 620, a display screen, and a user interface. As can be readily be appreciated, processor system 602, I/O interface 604, and memory system 606 can be implemented using any of a variety of components appropriate to the requirements of specific applications including (but not limited to) CPUs, GPUs, ISPs, DSPs, wireless modems (e.g., Wi-Fi, Bluetooth modems), serial interfaces, volatile memory (e.g., DRAM) and/or non-volatile memory (e.g., SRAM, and/or NAND Flash).

Memory system 606 is capable of storing various data, applications, and models. It is to be understood that the listed data, applications and models are a representative sample of what can be stored in memory and that various memory systems may store some or all of the various data, applications, and models listed. Further, any combination of data, applications, and models can be stored, and in some implementations, various data, applications, and/or models are stored temporarily. These data may include hemodynamic parameter data (e.g., blood pressure, heart rate), raw sensor data, motion data, historical trends, and metadata (e.g., timestamps, patient identifiers).

Hemodynamic monitoring system 600 can utilize a number of applications stored within memory system 606 to be executed by processor system 602 to perform a set of instructions, which can perform various computational methods as described herein. Applications that can be stored within a memory system 606 include a Real-time Hemodynamic Monitoring application 608, a Motion Detection application 610, and a Hemodynamic Data Correction application 612 for performing the various methods and processes described herein. The various applications can be provided as individual processes or as an ensemble of processes, each of which may be utilized to provide real-time continuous hemodynamic monitoring. Real-time hemodynamic data (e.g., corrected and/or sensed data) can also optionally be stored on memory system 606 and/or displayed on a display screen via the I/O interface 604. The Motion Detection application 610 may utilize accelerometer and/or gyroscope data to identify repositioning events. The Hemodynamic Data Correction application 612 may execute vCorr algorithms and/or reference-based adjustments (e.g., via virtual and/or physical heart reference sensors). Data may be visualized through the I/O interface 604 (e.g., on a local and/or remote display screen) and/or transmitted to a remote computing system.

While a specific hemodynamic monitoring system configuration is described above with reference to FIG. 6A, it should be readily appreciated that various hemodynamic monitoring systems utilized in the provision of hemodynamic monitoring can be implemented in any of a variety of configurations. Accordingly, the various systems and methods described herein should not be limited to any specific hemodynamic monitoring system configuration, but instead can be implemented using any variety of hemodynamic monitoring system configurations capable of sensing hemodynamic measurements.

FIG. 6B shows an example hemodynamic monitoring system 630 that integrates both pressure sensing and motion detection, according to some embodiments. The hemodynamic monitoring system 630 can correct for hydrostatic pressure shifts caused by changes in appendage orientation and/or elevation. The hemodynamic monitoring system 630 can combine signal inputs from a volume clamp 632 and an accelerometer 634. The hemodynamic monitoring system 630 can implement algorithmic corrections to yield a physiologically representative pressure output. The hemodynamic monitoring system 630 can include one or more features of the sensor system 101 and/or of the hemodynamic monitoring system 600.

The hemodynamic monitoring system 630 can include the volume clamp 632, which may be placed on a patient's appendage (e.g., finger, arm, toe, leg), to acquire continuous pressure waveforms. Additionally or alternatively to raw pressure measurements, the volume clamp 632 may generate data for beat detection 636. The beat detection 636 data may correspond to a timing and/or morphology of cardiac cycles of the patient's appendage. These beat detections may be used to derive additional hemodynamic features, such as virtual mean arterial pressure (vMAP) 640 and/or a stable virtual heart reference sensor (vHRS). The volume clamp 632 may be a part of and/or include one or more features of the cuff 20 and/or the pressurizable bladder 22 described above. The vMAP may be calculated using a trained machine learning model that takes in one or more variables as inputs, such as heart rate, cuff pressure values, and/or changes in pressure over time (e.g., max dP/dt).

The accelerometer 634 can be positioned in association with the clamp to generate motion signals associated with the volume clamp 632 (e.g., attached to the same appendage). The accelerometer 634 detects changes in orientation, inclination, or elevation of the appendage. The signal from the accelerometer 634 is passed to a motion detector 638, which determines when a significant motion event has occurred (e.g., a tilt of the surgical bed, lifting of the arm) that may introduce a hydrostatic gradient between the measurement site and heart level. When such an event is detected, the motion detector 638 issues a signal to the postprocessing unit 642. The accelerometer 634 may be a part of and/or include one or more features of the motion detection sensor (e.g., of the sensing system 12) described above.

The system 630 can include a postprocessing module 642. The postprocessing 642 can track vMAP over time and/or may use vMAP as an input. Additionally or alternatively, the postprocessing 642 may include correction logic for the vMAP. The postprocessing 642 may receive inputs from one or more of the beat detection 636 and/or the motion detector 638. Based on one or more of these inputs, the system can computes the vMAP 640, which can include an estimated mean arterial pressure of the patient, taking into account a height of the appendage above the heart. Additionally or alternatively, the system can estimate a virtual heart reference sensor (vHRS). The vHRS can represent an expected hydrostatic offset due to elevation or depression of the measurement site. In some embodiments, the vHRS may be held static between motion events, updating only upon verified repositioning, for example to reduce noise from physiological drift and/or transient anomalies.

The waveform correction module 644 can receive one or more pressure waveforms from the volume clamp 632 and/or the vHRS signal generated by the postprocessing 642. Based on one or more of these inputs, the module 644 applies a height-based correction to the raw pressure waveform, which may result in recalibrating the signal to reflect heart-level pressures. This correction step can improve the accuracy of pressure readings to better correspond to true physiological pressures even in response to a shift of the patient's appendage. The corrected pressure waveform can then be output at 646 as a refined signal to be displayed and/or for further processing. The output 646 can additionally or alternatively include a correction factor/offset described herein. The output 646 may be displayed on a display connected to the system 630 via a wired and/or wireless data interface.

FIG. 6C illustrates an example method 660 for correcting blood pressure measurements based on beat-level processing and motion detection, in accordance with some embodiments. The method 660 can be implemented by a hemodynamic monitoring system such as the hemodynamic monitoring system 630, the sensor system 101, and/or the hemodynamic monitoring system 600. As shown, the method may operate continuously or intermittently, for example in response to new cardiac beats identified from pressure waveform data acquired via a volume clamp placed on a patient's appendage.

At block 662, the system senses a new cardiac beat. The system may sense the new cardiac beat, for example, in response to a beat detection module that identifies the onset of a new heartbeat from the pressure waveform generated by a volume clamp. At block 664, the system estimates a vMAP for the new beat. The vMAP may correspond to an estimate of the patient's actual MAP, after compensating for a height of the appendage above (or below) the heart. In some embodiments, this vMAP may be generated by postprocessing logic (e.g., of the postprocessing 642) using one or more models, such as a machine learning model.

At block 666, the system determines whether to reject the vMAP value for this beat. For example, the system may compare the newly estimated vMAP to statistical norms, prior beats, or an ensemble of trained model predictions. If the vMAP falls outside of a defined confidence interval (e.g., exceeding a threshold for variability), the system may determine that the beat is noisy and/or otherwise unreliable and reject the vMAP value. If the system determines to reject the vMAP (path “true”), then at block 668, the system takes no corrective action for this beat and the method awaits the next sensed beat. In this way, noisy data are gated off to preserve the stability and reliability of downstream calculations.

If the vMAP is not rejected (path “false”), then the method proceeds to block 670, where the system determines whether a motion event has occurred. This determination may be based on motion signals received from an accelerometer and/or other motion detection module (e.g., accelerometer 634, motion detector 638), which may detect changes in orientation, inclination, and/or elevation of the pressure sensor relative to gravity. For example, the system may infer from the motion signals that the patient's arm has moved up or down, indicating a likely change in hydrostatic pressure relative to heart level.

If motion is detected (path “true”), then at block 672, the system updates the virtual heart reference sensor (vHRS). This vHRS may reflect a height offset based on the new position of the sensor and may be used to adjust measured pressures to reflect pressures at heart level. If no motion is detected (path “false”), the method 660 proceeds to block 674, where the system updates a bias term. The bias term may include one or more corrections learned from machine learning models. For example, this bias update may account for observed drifts in vMAP not attributable to motion, and may be used to adjust long-term signal trends. For example, the system may refine its internal model to distinguish between physiological changes and apparent changes due to slow sensor migration and/or calibration drift. In some embodiments, the system may repeat method 660 continuously, using incoming beats to update a stable and motion-aware pressure estimate in real time.

### Experimental Data

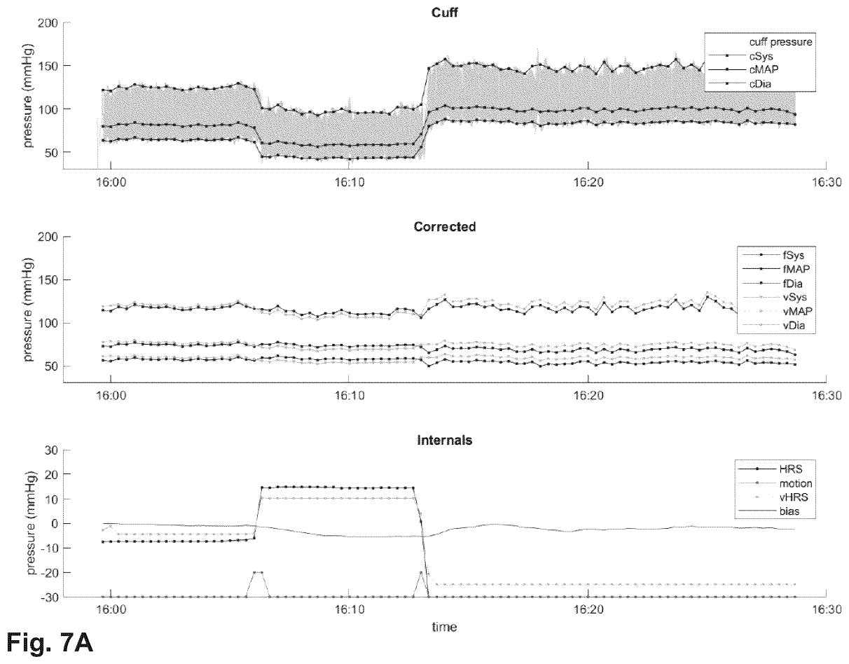

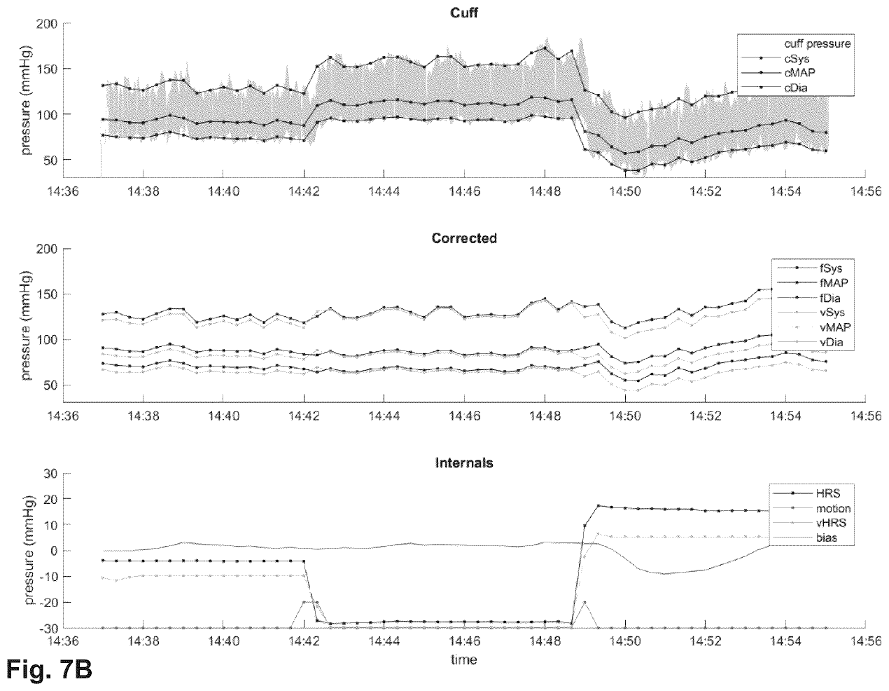

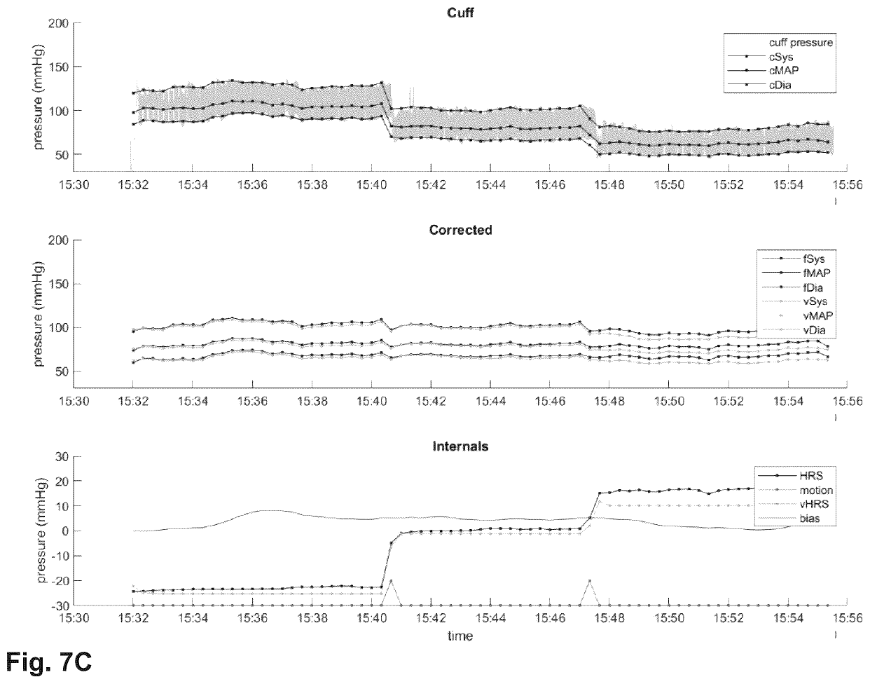

Provided in FIGS. 7A to 7C are examples of correcting sensed data utilizing a computational surrogate in accordance with various systems and methods described herein. Within each figure, the top panel depicts sensed arterial blood pressure obtained by a pressurizable cuff and photoplethysmograph via the volume clamp method. The bottom panel depicts detected motion of the site of sensing, correction via classical heart reference sensor, correction via a computational surrogate, and bias. The middle panel shows corrected sensed data, comparing corrections via the HRS system (darker shaded lines) with corrections via the computational surrogate (lighter shaded lines). As can be seen by the data results, the computational surrogate yields a correction that is very comparable to the classical HRS system. This data suggests that sensed hemodynamic parameters can successfully be corrected via a computational surrogate utilizing the systems and methods of the disclosure.

### EXAMPLE CLAUSES

1. A method for correcting sensed hemodynamic data in real-time, comprising:
   receiving, using hemodynamic monitoring system, sensor signals acquired from a sensor system upon a site of sensing;
   determining, using the hemodynamic monitoring system, a set of hemodynamic parameters derived from the sensor signals;
computing, using the hemodynamic monitoring system, a correction that accounts for vertical position of a sensor of the sensor system; and
continually correcting, using the hemodynamic monitoring system, a hemodynamic parameter using the correction that accounts for vertical position of the sensor.
2. The method of example 1, wherein computing, using the hemodynamic monitoring system, the correction that accounts for the vertical position of the sensor system further comprises:
   entering, using the hemodynamic monitoring system, a set of hemodynamic parameters into a trained computational model to yield a corrected hemodynamic parameter; and
   utilizing, using the hemodynamic monitoring system, the corrected hemodynamic parameter within an equation of invariant inputs to determine the correction that accounts for vertical position of the sensor system, wherein the invariant inputs include the corrected hemodynamic parameter and a sensed hemodynamic parameter, wherein the corrected hemodynamic parameter and the sensed hemodynamic parameter correspond to the same hemodynamic parameter.
3. The method of example 2, wherein the equation of invariant inputs is:

$$vHP + bias = sHP + vCorr$$

wherein vHP is the corrected hemodynamic parameter, sHP is a sensed hemodynamic parameter, and vCorr is the correction, and wherein vHP and psHP are the same hemodynamic parameter.
4. The method of example 3, wherein vHP is vMAP and sHP is sMAP.
5. The method of example 1, wherein computing the correction that accounts for vertical position of the sensor system further comprises:
   receiving, using the hemodynamic monitoring system, motion detector signals; and
   determining, using the hemodynamic monitoring system and based on the motion detector signals, that the correction corresponds to a change in vertical position of the sensor relative to a heart level.
6. The method of any one of examples 1 to 5, further comprising:
identifying, using the hemodynamic monitoring system, a vertical motion of the sensor system corresponding to a change in vertical position of the sensor system; and
updating, using the hemodynamic monitoring system, the correction that accounts for vertical position of the sensor system.
7. The method of example 6, wherein updating the correction that accounts for vertical position of the sensor system comprises:
   entering, using the hemodynamic monitoring system, an updated set of hemodynamic parameters into a trained computational model to yield an updated corrected hemodynamic parameter; and
   utilizing, using the hemodynamic monitoring system, the updated corrected hemodynamic parameter within an equation of invariant inputs to determine the correction that accounts for the vertical position of the sensor system, wherein the invariant inputs include the updated corrected hemodynamic parameter and an updated sensed hemodynamic parameter, wherein the updated corrected hemodynamic parameter and the updated sensed hemodynamic parameter correspond to the same hemodynamic parameter.
8. The method of example 6 or 7, wherein identifying the motion of the sensor system comprises:
receiving, using the hemodynamic monitoring system, motion detector signals, wherein the motion detector signals indicate that an amount of vertical motion is greater than a threshold.
9. The method of example 8, wherein the motion detector signals indicate that a change of gravitational force is greater than a threshold.
10. The method of example 8 or 9, wherein the motion detector signals are derived from a motion detector that is associated with the sensor system.
11. The method of example 6 or 7, wherein identifying the motion of the sensor system comprises: detecting, using the hemodynamic monitoring system, the vertical motion of the sensor system from a computational model trained to detect a change in sensed hemodynamic parameters in response to vertical repositioning of the sensor system.
12. The method of example 6, wherein updating the correction that accounts for vertical position of the sensor system comprises:
   receiving, using the hemodynamic monitoring system, motion detector signals; and
   determining, using the hemodynamic monitoring system and based on the motion detector signals, that the correction corresponds to the change of vertical position of the sensor relative to the heart level.
13. The method of any one of examples 1 to 12, wherein the sensor system comprises at least one of: an applanation tonometer, a pressure cuff, a photoplethysmograph, a peripheral arterial line, one or more electrode leads, a pulse oximeter, or a capacitance-based pressure sensor.
14. The method of any one of examples 1 to 13, wherein determining the set of hemodynamic parameters comprises:
   performing arterial tonometry, performing volume clamping, performing catheter-based hemodynamic monitoring, performing pulse wave transit time or pulse arrival time, performing photoplethysmography-based heart rate monitoring, or performing arterial pressure sensing via capacitance.
15. The method of any one of examples 1 to 14 further comprising:
   computing, using the hemodynamic monitoring system, a downstream hemodynamic parameter from a corrected sensed hemodynamic parameter.
16. The method of example 15, wherein the downstream hemodynamic parameter comprises a centralized hemodynamic parameter.
17. The method of example 16, wherein the centralized hemodynamic parameter comprises a blood pressure within the ascending aorta of a patient.
18. The method of any one of examples 1 to 17 further comprising:
   generating, using the hemodynamic monitoring system, a waveform utilizing corrected sensed hemodynamic parameters; or
generating, using the hemodynamic monitoring system, a waveform utilizing downstream hemodynamic parameters.
19. The method of example 18 further comprising:
   displaying the waveform on a display of the hemodynamic monitoring system.
20. The method of any one of examples 1 to 19, wherein the hemodynamic monitoring system comprises at least one of: a clinical use system, a portable system, or a wearable device.
21. The method of any one of examples 1 to 20, wherein the hemodynamic monitoring system excludes a paired sensor system for establishing vertical position of the sensing system relative to a heart based on a difference of vertical position between each sensor of the paired sensor system to compute the correction.
22. The method of any one of examples 1 to 21, wherein the hemodynamic monitoring system excludes one or more sensors positioned at heart level for establishing the vertical position of a heart to compute the correction.
23. The method of any one of examples 1 to 22, wherein the hemodynamic monitoring system excludes the use of a central blood pressure sensor positioned at a non-thoracic location to compute the correction.
The method of any one of examples 1 to 23, wherein the motion detector comprises at least one of an accelerometer or gyroscope configured to detect positional shifts of the sensor system.
24. The method of example 6 or 7, wherein identifying, using the hemodynamic monitoring system, a motion of the sensor system comprises:
receiving, using the hemodynamic monitoring system, motion detector signals, wherein the motion detector signals indicate that an amount of vertical motion is less than a threshold; and
determining, based on the signals indicating that the amount of vertical motion is less than the threshold, that the motion detector signals do not correspond to a change in the vertical position of the sensor system.
25. The method of any one of examples 1 to 24, wherein determining the set of sensed hemodynamic parameters comprises calculating a mean arterial pressure across a plurality of beats.
26. The method of example 8, wherein computing the correction that accounts for vertical position of the sensor system is based on receiving the motion detector signals indicating that the amount of vertical motion is greater than the threshold.
27. The method of any one of examples 1 to 25, further comprising:
determining that an override function is executed; and
delaying, in response to determining that the override function is executed, the continually correcting of the sensed hemodynamic parameter.
28. The method of example 27, wherein determining that the override function is executed comprises determining that a manual override selection is made.
29. The method of example 28, wherein determining that the manual override selection is made comprises determining that a button is selected.
30. The method of example 28, further comprising computing a confidence score of the correction.
31. A hemodynamic monitoring system for real-time correction of sensed hemodynamic data, the system comprising:
   a sensor system comprising a sensor; and
   a computational processing system in digital connection with the sensor system, the computational processing system comprising:
      a processor system; and
      a memory system comprising one or more applications that can direct the processor system to:
         receive sensor signals acquired from a sensor system upon a site of sensing;
      determine a set of hemodynamic parameters derived from the sensor signals;
         compute a correction that accounts for vertical position of the sensor system; and
         continually correct a sensed hemodynamic parameter using the correction that accounts for vertical position of the sensor.
32. The hemodynamic monitoring system of example 31, wherein one or more applications can further direct the processor system to:
   enter the set of sensed hemodynamic parameters into a trained computational model to yield a corrected hemodynamic parameter; and
   utilize the corrected hemodynamic parameter within an equation of invariant inputs to determine the correction that accounts for vertical position of the sensor system, wherein the invariant inputs include the corrected hemodynamic parameter and a sensed hemodynamic parameter, wherein the corrected hemodynamic parameter and the sensed hemodynamic parameter correspond to the same hemodynamic parameter.
33. The hemodynamic monitoring system of example 32, wherein the equation of invariant inputs is:

$$vHP + bias = sHP + vCorr$$

wherein vHP is the corrected hemodynamic parameter, sHP is a sensed hemodynamic parameter, and vCorr is the correction, and wherein vHP and sHP are the same hemodynamic parameter.
34. The hemodynamic monitoring system of example 33, wherein vHP is vMAP and sHP is sMAP.
35. The hemodynamic monitoring system of example 31, wherein one or more applications can further direct the processor system to:
receive motion detector signals, wherein the motion detector signals indicate a vertical position of the sensor system relative to heart level; and
   determine the correction using the vertical position of the sensor system relative to heart level.
36. The hemodynamic monitoring system of any one of examples 31 to 35, wherein one or more applications can further direct the processor system to:
identify a motion of the sensor system corresponding to a change in vertical position of the sensor system; and
update the correction that accounts for vertical position of the sensor system.
37. The hemodynamic monitoring system of example 36, wherein one or more applications can further direct the processor system to:
   enter an updated set of sensed hemodynamic parameters into a trained computational model configured to yield an updated corrected hemodynamic parameter; and
   utilize the updated corrected hemodynamic parameter within an equation of inputs to determine a correction that accounts for vertical position of the sensor system, wherein the inputs include the updated corrected hemodynamic parameter and an updated sensed hemodynamic parameter, wherein the updated corrected hemodynamic parameter and the updated sensed hemodynamic parameter are the same hemodynamic parameter.
38. The hemodynamic monitoring system of example 36 or 37 further comprising a motion detector, wherein one or more applications can further direct the processor system to:
receive motion detector signals, wherein the motion detector signals indicate that an amount of vertical motion is greater than a threshold.
39. The hemodynamic monitoring system of example 31, wherein the motion detector signals indicate that a change of gravitational force is greater than a threshold.
40. The hemodynamic monitoring system of example 38 or 39, wherein the motion detector is associated with the sensor system.
41. The hemodynamic monitoring system of example 39 or 40, wherein one or more applications can further direct the processor system to:
   detect vertical motion of the sensor system from a computational model trained to detect a change in sensed hemodynamic parameters in response to vertical repositioning of the sensor system.
42. The hemodynamic monitoring system of example 29 further comprising a motion detector, wherein one or more applications can further direct the processor system to:
receive motion detector signals, wherein the motion detector signals indicate a change in vertical position of the sensor system relative to heart level; and
   determine the correction using the change of vertical position of the sensor system relative to heart level.
43. The hemodynamic monitoring system of any one of examples 31 to 42 wherein the sensor system comprises at least one of: an applanation tonometer, a pressure cuff, a photoplethysmograph, a peripheral arterial line, one or more electrode leads, a pulse oximeter, or a capacitance-based pressure sensor.
44. The hemodynamic monitoring system of any one of examples 31 to 43, wherein set of sensed hemodynamic parameters is determined by at least one of: arterial tonometry, volume clamping, catheter-based hemodynamic monitoring, pulse wave transit time or pulse arrival time, photoplethysmography-based heart rate monitoring, and arterial pressure sensing via capacitance.
45. The hemodynamic monitoring system of any one of examples 31 to 44, wherein one or more applications can further direct the processor system to:
   compute a downstream hemodynamic parameter from a corrected sensed hemodynamic parameter.
46. The hemodynamic monitoring system of example 45, wherein the downstream hemodynamic parameter is a centralized hemodynamic parameter.
47. The hemodynamic monitoring system of example 46, wherein the centralized hemodynamic parameter is blood pressure within the ascending aorta of a patient.
48. The hemodynamic monitoring system of any one of examples 31 to 47 wherein one or more applications can further direct the processor system to:
   generate a waveform utilizing corrected sensed hemodynamic parameters; or generate a waveform utilizing downstream hemodynamic parameters.
49. The hemodynamic monitoring system of example 48 further comprising a display, wherein one or more applications can further direct the processor system to:
   display the waveform on the display.
50. The hemodynamic monitoring system of any one of examples 31 to 49, wherein the hemodynamic monitoring system is at least one of: a clinical use system, a portable system, or a wearable device.
51. The hemodynamic monitoring system of any one of examples 31 to 50, wherein the hemodynamic monitoring system excludes a paired sensor system for establishing vertical position of the sensing system relative to a heart based on a difference of vertical position between each sensor of the paired sensor system to compute the correction.
52. The hemodynamic monitoring system of any one of examples 31 to 51, wherein the hemodynamic monitoring system excludes one or more sensors positioned at heart level for establishing the vertical position of a heart to compute the correction.
53. The hemodynamic monitoring system of any one of examples 31 to 52, wherein the hemodynamic monitoring system excludes the use of a central blood pressure sensor positioned at a non-thoracic location to compute the correction.

What is claimed is:

1. A hemodynamic monitoring system for real-time correction of peripherally sensed blood pressure data, the system comprising:
a display screen;
a first sensor system comprising a blood pressure sensor configured to be attached to a wrist or hand of a patient;
a second sensor system comprising a motion sensor configured to detect displacement of the first sensor system in a vertical direction; and
a computational processing system in digital connection with the first and second sensor systems, the computational processing system comprising:
a processor system; and
a memory system comprising one or more applications that can direct the processor system to:
receive a BP waveform signal acquired from the first sensor system;
determine a set of hemodynamic parameters derived from the BP waveform signal, including a sensed blood pressure parameter;
enter the set of sensed hemodynamic parameters into a trained computational model to yield a corrected blood pressure parameter that accounts for a change in a vertical position of the first sensor system;
detecting a motion of the first sensor system using the second sensor, the motion having a magnitude above a threshold in the vertical direction;
correcting the sensed hemodynamic parameter using the corrected blood pressure parameter based on having detected the motion of the first sensor system; and
displaying the corrected blood pressure parameter on the display screen.

2. The system of claim 1, wherein the sensed blood pressure parameter and the corrected blood pressure parameters are the same parameter and are one of: mean arterial pressure (MAP), systolic pressure, and diastolic pressure.

3. The system of claim 1, wherein the memory system is further configured to adjust the BP waveform signal based on the corrected blood pressure parameter to correct for the change in the vertical position of the first sensor system.

4. A method for correcting sensed hemodynamic data in real-time, comprising:
receiving, using hemodynamic monitoring system, sensor signals acquired from a sensor system upon a site of sensing;
determining, using the hemodynamic monitoring system, a set of hemodynamic parameters derived from the sensor signals;
computing, using the hemodynamic monitoring system, a correction that accounts for vertical position of a sensor of the sensor system; and
continually correcting, using the hemodynamic monitoring system, a hemodynamic parameter using the correction that accounts for vertical position of the sensor.

5. The method of claim 4, wherein computing, using the hemodynamic monitoring system, the correction that accounts for the vertical position of the sensor system further comprises:
entering, using the hemodynamic monitoring system, a set of hemodynamic parameters into a trained computational model to yield a corrected hemodynamic parameter; and
utilizing, using the hemodynamic monitoring system, the corrected hemodynamic parameter within an equation of invariant inputs to determine the correction that accounts for vertical position of the sensor system, wherein the invariant inputs include the corrected hemodynamic parameter and a sensed hemodynamic parameter, wherein the corrected hemodynamic parameter and the sensed hemodynamic parameter correspond to the same hemodynamic parameter.

6. The method of claim 5, wherein the equation of invariant inputs is:
$$vHP + bias = sHP + vCorr$$
wherein vHP is the corrected hemodynamic parameter, sHP is a sensed hemodynamic parameter, and vCorr is the correction, and wherein vHP and psHP are the same hemodynamic parameter.

7. The method of claim 6, wherein vHP is vMAP and sHP is sMAP.

8. The method of claim 4, wherein computing the correction that accounts for vertical position of the sensor system further comprises:
receiving, using the hemodynamic monitoring system, motion detector signals; and
determining, using the hemodynamic monitoring system and based on the motion detector signals, that the correction corresponds to a change in vertical position of the sensor relative to a heart level.

9. The method of claim 8, further comprising:
identifying, using the hemodynamic monitoring system, a vertical motion of the sensor system corresponding to a change in vertical position of the sensor system; and
updating, using the hemodynamic monitoring system, the correction that accounts for vertical position of the sensor system.

10. The method of claim 9, wherein updating the correction that accounts for vertical position of the sensor system comprises:
entering, using the hemodynamic monitoring system, an updated set of hemodynamic parameters into a trained computational model to yield an updated corrected hemodynamic parameter; and
utilizing, using the hemodynamic monitoring system, the updated corrected hemodynamic parameter within an equation of invariant inputs to determine the correction that accounts for the vertical position of the sensor system, wherein the invariant inputs include the updated corrected hemodynamic parameter and an updated sensed hemodynamic parameter, wherein the updated corrected hemodynamic parameter and the updated sensed hemodynamic parameter correspond to the same hemodynamic parameter.

11. The method of claim 9, wherein identifying the motion of the sensor system comprises:
receiving, using the hemodynamic monitoring system, motion detector signals, wherein the motion detector signals indicate that an amount of vertical motion is greater than a threshold.

12. The method of claim 9, wherein identifying the motion of the sensor system comprises:
detecting, using the hemodynamic monitoring system, the vertical motion of the sensor system from a computational model trained to detect a change in sensed hemodynamic parameters in response to vertical repositioning of the sensor system.

13. The method of claim 4, wherein determining the set of hemodynamic parameters comprises:
performing arterial tonometry, performing volume clamping, performing catheter-based hemodynamic monitoring, performing pulse wave transit time or pulse arrival time, performing photoplethysmography-based heart rate monitoring, or performing arterial pressure sensing via capacitance.

14. The method of claim 4, further comprising:
generating, using the hemodynamic monitoring system, a waveform utilizing corrected sensed hemodynamic parameters; or
generating, using the hemodynamic monitoring system, a waveform utilizing downstream hemodynamic parameters.
